(19) United States
(12) Patent Application Publication
(10) Pub. No.: US 2022/0331483 A1
(43) Pub. Date: Oct. 20, 2022

(54) NERVE REGENERATION INDUCING MATERIAL

(71) Applicants: CYFUSE BIOMEDICAL K.K., Tokyo (JP); Shinshu University National University Corporation, Matsumoto-shi, Nagano (JP)

(72) Inventors: Tetsuya IMAMURA, Nagano (JP); Mitsuru SHIMAMURA, Tokyo (JP)

(73) Assignees: CYFUSE BIOMEDICAL K.K., Tokyo (JP); Shinshu University National University Corporation, Matsumoto-shi, Nagano (JP)

(21) Appl. No.: 17/611,472

(22) PCT Filed: May 14, 2020
(86) PCT No.: PCT/JP2020/019234

§ 371 Date: Jun. 29, 2022

(30) Foreign Application Priority Data
May 16, 2019 (JP) 2019-093184

(51) Int. Cl. A61L 27/36

(52) U.S. Cl. CPC A61L 27/36

(57) ABSTRACT

A material for inducing nerve regeneration in a transplantation site, or a material for recovering the function of nerve tissues in a transplantation site, the material comprising a cell structure having a thickness of at least 300 μm that is constructed by stacking the spheroids of bone marrow-derived cells, adipose tissue-derived cells, dental pulp-derived cells, amnion-derived cells, placenta-derived cells, umbilical cord-derived cells, or umbilical cord blood-derived cells, on a needle-shaped body arranged on a substrate.

### TECHNICAL FIELD

The present invention relates to a material for inducing nerve regeneration in a transplantation site, wherein the material comprises a three-dimensional cell structure having a thickness of at least 300 μm.

### BACKGROUND ART

Previously, a poorly compliant bladder and a cystatrophia animal model have not been established in the fundamental research area of urology. On the other hand, a radiation-injured bladder model similar to such a poorly compliant bladder or cystatrophia has been established by applying radioactive rays to a rat bladder. In addition, regarding a freeze-injured bladder model, the problem still remains that the freeze-injured bladder model is reversible and thus, an injured site is recovered as a result of natural healing.

The poorly compliant bladder or cystatrophia is irreversible. Hence, when an irreversible damage is given to the bladder by irradiation, smooth muscle cells are reduced and also, neurons are significantly reduced from the tissues of the radiation-injured bladder model, as in the case of freezing injury (Non Patent Literature 1).

### CITATION LIST

### Non Patent Literature

Non Patent Literature 1: Imamura, Ishizuka, Zhang, Hida, Gautam, Kato, Nishizawa. Bone marrow-derived cells implanted into radiation-injured urinary bladders reconstruct bladder tissues in rats. Tissue Engineering Part A, 18: 1698-1709, 2012

### SUMMARY OF INVENTION

### Technical Problem

In the present invention, it has been desired to develop a regenerative medicine material, which is capable of recovering a reduction in neurons associated with a poorly compliant bladder or cystatrophia.

As a result of intensive studies directed towards achieving the aforementioned object, the present inventor has found that a three-dimensional cell structure having a thickness of at least 500 μm functions as a tissue regeneration-inducing material in the transplantation site, thereby completing the present invention.

### Solution to Problem

Specifically, the present invention is as follows.

(1) A material for inducing nerve regeneration in a transplantation site, wherein the material comprises a cell structure having a thickness of at least 300 μm that is constructed by stacking the spheroids of bone marrow-derived cells, adipose tissue-derived cells, dental pulp-derived cells, amnion-derived cells, placenta-derived cells, umbilical cord-derived cells, or umbilical cord blood-derived cells, on a needle-shaped body arranged on a substrate.
(2) The nerve regeneration-inducing material according to the above (1), wherein the transplantation site is a tissue or an organ comprising a smooth muscle layer.
(3) The nerve regeneration-inducing material according to the above (2), wherein the tissue or organ comprising a smooth muscle layer is at least one selected from the group consisting of bladder, ureter, urethra, penis, uterus, vagina, spermatic duct, and fallopian tube.
(4) The nerve regeneration-inducing material according to any one of the above (1) to (3), which further provides at least one selected from the group consisting of regeneration of microvessels, normalization of collagen fibers, the improvement of hypoxia, and the improvement of basal pressure, threshold pressure, micturition pressure, voiding interval, micturition volume, and residual volume in the bladder.
(5) A material for recovering nerve function in a transplantation site, wherein the material comprises a cell structure having a thickness of at least 300 μm that is constructed by stacking the spheroids of bone marrow-derived cells, adipose tissue-derived cells, dental pulp-derived cells, amnion-derived cells, placenta-derived cells, umbilical cord-derived cells, or umbilical cord blood-derived cells, on a needle-shaped body arranged on a substrate.
(6) The function-recovering material according to the above (5), wherein the transplantation site is a tissue or an organ comprising a smooth muscle layer.
(7) The function-recovering material according to the above (6), wherein the tissue or organ comprising a smooth muscle layer is at least one selected from the group consisting of bladder, ureter, urethra, penis, uterus, vagina, spermatic duct, and fallopian tube.
(8) The function-recovering material according to the above (6) or (7), which further provides at least one selected from the group consisting of regeneration of microvessels, normalization of collagen fibers, the improvement of hypoxia, and the improvement of basal pressure, threshold pressure, micturition pressure, voiding interval, micturition volume, and residual volume in the bladder.

### Advantageous Effects of Invention

According to the present invention, a material for inducing nerve regeneration in a transplantation site is provided. When the nerve regeneration-inducing material of the present invention is transplanted into a target tissue or organ, nerve regeneration that provides the recovery of the function of the tissue or organ can be induced in the transplantation site.

### BRIEF DESCRIPTION OF DRAWINGS

FIG. 1 includes a view of the construction of a bone marrow cell-derived cell structure, and a view showing the transplantation thereof. Panel A is a photograph showing that bone marrow-derived cell spheroids formed from 4.0×10⁴ cells were stacked on an array consisting of 9×9 microneedles (approximately 5×5 mm seen from the above), and was assembled. Panel B is a photograph of the stacking of bone marrow-derived cell spheroids (three layers, approximately 1 mm), which is seen from the side. Panel C shows a bone marrow-derived cell structure, which was obtained after the stacked spheroids were subjected to a perfusion culture for 7 days and were then removed from the microneedle array. The size of the cell structure was approximately 3×3 mm, and the height thereof was 1 mm. Panel D is a photograph showing that the circumference of the bladder of a recipient rat was excised 2 weeks after the final radiotherapy. Panel E is a photograph showing that the anterior wall of the urinary bladder irradiated with radioactive rays was excised by approximately 5 mm (two-headed arrow). Panel F is a photograph showing that a cell structure (asterisk) was transplanted into the excised portion of the anterior wall of the urinary bladder irradiated with radioactive rays, and was then fixed with silk sutures 7-0. Panel G is a photograph showing that the transplantation site of the cell structure was covered with an absorbent hemostatic plug (arrowhead).

FIG. 2 is a view showing the results of the histological staining of the cell structure of the present invention. Panel A is a photograph showing that there were no disordered defects in the center of the structure as a result of observation by Hematoxylin Eosin (HE) staining. Panel B is a view showing that the gathered spheroids were allowed to come into contact with one another via proliferating cells and were fused with one another (Masson's trichrome staining). Panel C is a partially enlarged view of Panel A. Even looking at the enlarged view, there were no disorder defects in the center of the structure. Panel D is a photograph showing that, in continuous sections from an identical area, the spheroids were allowed to come into contact with one another via extracellular matrixes secreted from the bone marrow-derived cells (Masson's trichrome staining, arrowheads).

FIG. 3 is a view showing the histological test results of bladders into which the cell structure of the present invention was not transplanted (n=6) and bladders into which the cell structure of the present invention was transplanted (n=6), which were obtained 2 weeks after the transplantation. Panels A, B and C are photographs showing the bladders into which the cell structure of the present invention was not transplanted (i.e., bladders subjected to a control surgery of a false structure). Panel A shows that there was no clear natural healing of the wounds regarding incisions formed on the anterior wall of the urinary bladder (large arrowheads). Panel B shows that there were a plurality of inflammatory cells in the wound sites (small arrowheads). Panel C shows that the smooth muscle layer was thin in the wound area and the distribution thereof was sparse (black asterisks). Panel D, E and F are photographs showing the bladders into which the cell structure of the present invention was transplanted. The white arrows shown in Panels D-F each show the wound made with silk sutures 7-0. Panel D shows that the transplanted cell structure (black arrow) was clearly present in the anterior wall of the urinary bladder. Panel E shows that the transplanted cell structures (black arrows) survived, and that there were almost no inflammatory cells in the bladder tissues near the transplanted region. Panel F shows that smooth muscle layers (black asterisks) were reconstructed in the bladder tissues near the transplanted cell structures Panel G is a view showing that the transplanted structures had blood vessels in the box shown in Panel F. Panel H shows that smooth muscle cells comprising blood vessels were negative to the green fluorescent protein (GFP) antibody. The blood vessels in the transplanted cell structures were blood vessels extended from the recipient tissues (arrows). Bone marrow-derived cells that constituted the cell structures were detected by the GFP antibody (green). Panel I shows that the cells in the transplanted cell structures were positive to a smooth muscle actin (SMA) antibody (red). The blood vessels are indicated with the arrow portions. Panel J is a view showing double positive cells (yellow) that were positive to the GFP antibody (green) and the SMA antibody (red). The transplanted cells were differentiated into smooth muscle cells. The blow portion indicates a nucleus, and the blood vessels are indicated with the arrow portions.

FIG. 4 is a view showing the histological test results of bladders into which the cell structure of the present invention was not transplanted (n=6) and bladders into which the cell structure of the present invention was transplanted (n=4), which were obtained 4 weeks after the transplantation. Panels A, B and C are photographs showing the bladders into which the cell structure of the present invention was not transplanted (i.e., bladders subjected to a control surgery of a false structure). The white asterisks in Panel A, B and C indicate that adipose tissues could not be removed and were adhered. Panel A shows that the excised portions were not completely healed (large arrowheads). Panel B shows that there were a large number of inflammatory cells (small arrowheads) in the wound area. Panel C shows that the smooth muscle layers were disordered in the wound area. Panel D, E and F show the bladders into which the cell structure of the present invention was transplanted. In Panel D, the transplanted cell structure (black arrow) is easily observed on the anterior wall of the urinary bladder. The transplanted cell structure (black arrow) is fully integrated with the bladder wall of the recipient. Panel E shows that the cell structures of the present invention (black arrows) transplanted into the bladder were histologically intact and had almost no inflammatory cells. Panel F shows that smooth muscle layers (black asterisks) were reconstructed in bladder tissues near the transplanted region, as in the case of the bladder tissues 2 weeks after the transplantation. The white arrows shown in Panels B-F each show the wound made with silk sutures 7-0. Panels G, H and I are photographs showing that the box a in Panel F was fluorescently stained. Among the cell structures of the present invention transplanted into the irradiated bladder, GFP-positive (Panel G: green) transplanted cells were differentiated into SMA-positive (Panel H: red) smooth muscle cells, and the smooth muscle cells and receptive tissues surrounding blood vessels (asterisks) formed a cluster (Panel I: yellow). The blue shown in Panel I indicates nuclear staining. Panel J, K and L are photographs showing that the box b in Panel F was fluorescently stained. Near the outer edge of the transplanted structure, GFP-positive (Panel J: green) transplanted cells were differentiated into SMA-positive (Panel K: red) smooth muscle cells, and the smooth muscle cells formed a cluster (Panel L: yellow). The blue shown in FIG. 4L indicates nuclear staining.

FIG. 5 includes photographs showing that nerve fibers were reconstructed in the incision boundary portion of a control group subjected to a sham surgery and in recipient bladder tissues into which the cell structure of the present invention was transplanted. Panel A shows that almost no acetylcholinesterase-positive cells were present in the bladders at the time of 2 weeks after the sham surgery as a control (n=6). Panel B shows that, in contrast to the photograph of Panel A, when the recipient bladder tissues 2 weeks after the surgery were subjected to immunofluorescence staining, several calcitonin gene-related peptide (CGRP)-positive afferent neurons were present (Panel B: red, white arrowheads). Panels C and D show that the recipient bladders 2 weeks after the transplantation of the cell structures (n=6) had several acetylcholinesterase-positive cells (Panel C: brown staining, black arrow) and CGRP-positive cells (Panel D: red, white arrows). Panels E and F show that acetylcholinesterase-positive cells were not present in the bladders 4 weeks after the sham surgery (n=6), as with 2 weeks after the sham surgery (Panel E), and that CGRP-positive cells were also present at the same level as, or at a level smaller than 2 weeks after the sham surgery. The surgery (F: red, white arrow). Panels G and H are photographs showing the bladders 4 weeks after the transplantation of the cell structures (n=4). Panel G shows that a larger amount of acetylcholinesterase (G: deep brown staining, black arrows) was comprised in the recipient bladder than the second week after the transplantation. Panel H shows that a larger number of CGRP-positive cells (Panel H: red, white arrows) were observed compared with those 2 weeks after the transplantation of the cell structures.

FIG. 6 includes photographs showing the arrangement of collagen fibers in the recipient bladders, and the arrangement of P4HB-positive cells catalyzing the proline hydrolysis of collagen components and (HIF1α-positive) cells expressing HIF1α transcriptional factors as hypoxia inducible factors. Panel A is a photograph showing that the bladder tissues 2 weeks after a sham surgery (control bladders, n=6) were stained with Sirius Red. The asterisks indicate collagen fibers. Panel B shows that collagen fibers (Panel A, asterisks) were infiltrated into extracellular matrix surrounding an injured smooth muscle layer. In the collagen fiber region, cells expressing P4HB (green, arrows) serving as fibrosis markers were present. Panel C shows that a large number of HIF1α-positive cells serving as hypoxia markers were present in a control bladder group (green, arrows). Panel D shows that collagen fibers (asterisks) were distributed among the clusters of the reconstructed smooth muscle cells 2 weeks after the transplantation of the cell structures of the present invention (n=6). Panels E and F show that small numbers of P4HB-positive cells (Panel E: green, arrows) and HIF1α-positive cells (Panel F: green, arrows) were present even in the clusters of smooth muscle cells. Panel G shows that collagen fibers (asterisks) were significantly enlarged 4 weeks after a control sham surgery (n=6), and that a large amount of extracellular matrix was present in the absence of a smooth muscle layer. Panels H and I show that only a small amount of smooth muscle layer (red) was present 4 weeks after the control sham surgery (n=6), whereas a large number of P4HB-positive cells (Panel H: green, arrows) and a large number of HIF1α-positive cells (Panel I: green, arrows) were present. Panel J shows that collagen fibers (asterisks) were distributed among the clusters of the smooth muscle cells in the recipient bladders (n=4) 4 weeks after the transplantation of the cell structures, as with those 2 weeks after the transplantation of the cell structures. In Panels K and L, only small numbers of P4HB-positive cells (Panel K: green, arrows) and HIF1α-positive cells (Panel L: green, arrows) were present in the clusters of smooth muscle cells in bladder tissues in the case of the recipient bladders (n=4) 4 weeks after the transplantation of the cell structures. The red in Panels K and L indicates SMA-positive smooth muscle cells, and the blue indicates a cell nucleus.

FIG. 7 is a view showing the micturition patterns of sham surgery control rats (n=6) and cell structure-transplanted rats (n=4), 4 weeks after the surgery. The sham surgery control rats (Panel A) exhibited symptoms apparently showing frequent micturition, such as irregular voiding intervals of less than 5 minutes (upper trace: arrowheads, micturition points) and a micturition volume of less than 1 ml (lower trace). In contrast, in the cell structure-transplanted rats (Panel B), the voiding interval was approximately 5 minutes (upper trace: arrowheads, micturition points), the micturition volume was approximately 1 ml (lower trace), and thus, it was demonstrated that the rats urinated regularly.

FIG. 8 is a view showing the results obtained by comparing micturition parameters between sham surgery control rats (n=6 in the entire period) and cell structure-transplanted rats (n=6 for 2 weeks; n=4 for 4 weeks). The sham surgery control rats are indicated with white bar graphs, whereas the cell structure-transplanted rats are indicated with gray bar graphs. The case of being significant (P<0.05) compared with the sham surgery control group is indicated with the asterisk (*). In addition, a significant difference between 4 weeks after the surgery and 2 weeks after the surgery is indicated with the dagger (†) (†P<0.05, ††P<0.01). Two weeks after the surgery, there was no difference between the two groups, in terms of basal pressure (Panel A), threshold pressure (Panel B), micturition pressure (Panel C), voiding interval (Panel D), and micturition volume (Panel E). Panel F shows that the residual volume (Panel F) 2 weeks after the transplantation of the structures was significantly lower than that in the controls in the same period. Four weeks after the sham surgery, the basal pressure (Panel A), the threshold pressure (Panel B), the micturition pressure (Panel C), and the residual volume (Panel F) were not changed to such an extent that a significant difference was generated from each value 2 weeks after the surgery. The voiding interval (Panel D) and the micturition volume (Panel E) were significantly lower than individual values 2 weeks after the surgery. Four weeks after the transplantation of the cell structures, all of the micturition parameters were not significantly changed from the values 2 weeks after the transplantation of the structures. Four weeks after the sham surgery or the transplantation of the cell structures, there was no significant difference between the sham surgery group and the cell structure transplantation group, in terms of the basal pressure (Panel A), the threshold pressure (Panel B), and the micturition pressure (Panel C). However, both the voiding interval (Panel D) and the micturition volume (Panel E) in the cell structure-transplanted rats were significantly higher than the voiding interval and the micturition volume in the control rats (dagger symbols). Moreover, the residual volume (Panel F) in the cell structure-transplanted rats was lower than the residual volume in the control rats.

### DESCRIPTION OF EMBODIMENTS

The present invention relates to a material for promoting regeneration of the nerve in a tissue or an organ of interest by transplantation of a bone marrow-derived cell structure into the tissue or organ of interest. In the present invention, it is also possible to use adipose tissue-derived cells, dental pulp-derived cells, amnion-derived cells, placenta-derived cells, umbilical cord-derived cells, or umbilical cord blood-derived cells, instead of the bone marrow-derived cells. The present invention relates to a material for recovering nerve tissues or a material for improving the function of nerve tissues, wherein the material comprises a cell structure having a thickness of at least 300 μm that is constructed by stacking the spheroids of bone marrow-derived cells, adipose tissue-derived cells, dental pulp-derived cells, amnion-derived cells, placenta-derived cells, umbilical cord-derived cells, or umbilical cord blood-derived cells, on a needle-shaped body arranged on a substrate.

The present inventor has attempted to transplant a bone marrow-derived cell structure into an injured bladder, so as to regenerate functional bladder tissues.

### 1. Production of Cell Structure

### (1-1) Cells

The cells used to produce the present cell structure are, for example, bone marrow-derived cells. Bone marrow-derived cells means cells obtained by subjecting cells collected from the bone marrow to a primary culture in a collagen-coated culture plate, then adhering and extending the cells in the culture plate, and then allowing the cells to proliferate. Such marrow-derived cells may be either a mixture of multiple types of cells that mainily include mesenchymal cells comprising stem cells, or cells separated by a cell sorter using multiple cell markers, etc. In the present invention, it is also possible to use adipose tissue-derived cells, dental pulp-derived cells, amnion-derived cells, placenta-derived cells, umbilical cord-derived cells, or umbilical cord blood-derived cells, instead of the bone marrow-derived cells.

The above-described bone marrow-derived cells, adipose tissue-derived cells, dental pulp-derived cells, amnion-derived cells, placenta-derived cells, umbilical cord-derived cells, or umbilical cord blood-derived cells are cultured or maintained in a medium suitable for individual types of cells, and the cells are prepared at time of use. Besides, various types of antibiotics, fetal bovine serum, and the like can be added to the medium, as necessary.

Thus, when the bone marrow-derived cells are continuously cultured, the cells are aggregated to form a cell aggregate, namely, a spheroid. The ability of the cells to form a spheroid can be examined, for example, by a morphological examination using an optical microscope.

### (1-2) Method for Producing Three-Dimensional Structure

A method of arranging the cells in any given three-dimensional space to produce a three-dimensional structure of the cells has been known (WO2008/123614). This method comprises arranging a needle-shaped body in the shape of a needle point holder on a substrate, and then sticking a cell mass into the needle-shaped body.

In the present invention, by utilizing the above-described method, spheroids are stacked to produce a three-dimensional cell structure (three-dimensional structure). Since an automatic stacking robot has already been known to realize the above-described method (Bio 3D Printer “Regenova” (registered trademark), CYFUSE BIOMEDICAL K.K.), the three-dimensional structure is preferably produced using this robot.

The number of spheroids arranged and the shape of spheroids arranged are not particularly limited, and these are arbitrarily determined.

In addition, the thickness of a cell structure produced is set to be, at least, 300 μm. The thickness of the obtained structure is, for example, 300 μm to 1800 μm, 500 μm to 1500 μm, 600 μm to 1200 μm, or 600 μm to 1800 μm. By setting the thickness of the cell structure within the aforementioned range, not only paracrine effects are obtained upon the transplantation of the bone marrow cells, but also, blood vessels are induced to the transplanted tissues so as to reduce a fibrotic lesion, and further, the transplanted bone marrow cells are directly differentiated into tissues that constitute bladder tissues. It is expected that the improvement of a fibrotic lesion and reconstruction of bladder tissues will progress integrally. With regard to adipose tissue-derived cells, dental pulp-derived cells, amnion-derived cells, placenta-derived cells, umbilical cord-derived cells, and umbilical cord blood-derived cells, the number of cells arranged and the shape of the cells arranged can be set to be the same as those for the marrow-derived cells.

The three-dimensional cell structure that is formed from bone marrow-derived cells, adipose tissue-derived cells, dental pulp-derived cells, amnion-derived cells, placenta-derived cells, umbilical cord-derived cells, or umbilical cord blood-derived cells, as described above, is also referred to as “the cell structure of the present invention.”

### 2. Transplantation of Cell Structure

Next, the cell structure of the present invention is transplanted into the tissue or organ of a recipient patient (subject animal).

The transplantation site is not particularly limited, as long as it is a tissue or an organ, in which regeneration of the nerve is desired. In addition, the transplantation method is not particularly limited, and any given method is applied herein.

The tissue or organ as a target of the transplantation of the cell structure of the present invention is a tissue or an organi comprising a smooth muscle layer. Examples of such a tissue or an organ may include bladder, ureter (upper urinary tract), urethra (lower urinary tract), penis, uterus, vagina, spermatic duct, and fallopian tube.

### 3. Application

As described above, when the cell structure of the present invention is transplanted into a transplantation site of interest, a smooth muscle layer is reconstructred in the transplantation site, and at the same time, the nerve is regenerated.

After completion of the transplantation, whether or not a predetermined nerve is regenerated is confirmed. In the case of a human, for example, this confirmation is carried out by performing a non-invasive urodynamic test, so that the recovery of bladder functions associated with nerve regeneration can be clarified. When peripheral nerves that govern the functions of the bladder (wherein the sympathetic nerve is a hypogastric nerve, the parasympathetic nerve is a pelvic nerve, and the somatic nerve is a pudendal nerve) are regenerated, appropriate collection of urine and micturition can be carried out. Specifically, the bladder is extended (relaxed), the urethra is contracted, and the amount of urine collected is increased at the time of first desire to urinate (i.e., a desire to urinate for feeling that a certain amount of urine is accumulated in the bladder, and that urine can be retained for a while), and involuntary contraction of the detrusor muscle (i.e., bladder contraction that is regardless of one's intention) is disappeared, so that sufficient collection of urine (250 ml or more) can be carried out and thus, micturition can be voluntarily carried out. Moreover, upon micturition, the bladder is contracted, and the urethra is relaxed, so that the collected urine can be completely urinated without interruption of the micturition. That is to say, nerve regeneration can be evaluated by confirming whether or not the bladder and the urethra cooperatively perform conflicting movements and as a result, appropriate urine collection and micturition can be carried out.

### (3-1) Regeneration of Microvessels

The cell structure of the present invention is transplanted into a bladder that has been injured by irradiation, and infiltration of vascular endothelial cells is thereby caused to the recipient, so that microvessels can be regenerated.

### (3-2) Improvement of Hypoxia

When the cell structure of the present invention is transplanted into tissues that are under hypoxic conditions, the cells are engrafted, so that the hypoxic conditions of the recipient tissues can be improved. Accordingly, the cell structure of the present invention can be applied to a pathologic condition, in which a fiber layer is formed on histological tissues as a result of proliferation and/or accumulation of fibroblasts or fibroblast-like tissues and the cells constituting the tissues are under hypoxic conditions.

### (3-3) Normalization of Collagen Fibers

When the cell structure of the present invention is transplanted to a disease exhibiting such findings that a collagen layer, in which collagen-generating cells are detected by histopathology or a non-invasive in vivo imaging technique, is broken, swollen, enlarged or scattered, the cells are engrafted, so that the collagen fibers of the recipient tissues can be normalized.

### (3-4) Regeneration of Nerve Tissues

When the cell structure of the present invention is transplanted to a disease exhibiting such findings that a nerve, for example, a peripheral nerve disappears, the cells are engrafted, neurons are infiltrated into the recipient tissues, and the functions are thereby recovered. This recovery can be observed by histopathology or a non-invasive in vivo imaging technique.

### (3-5) Diagnostic Marker Used in Treatment of Fibrosis

Cells that express a PH4B protein serving as a fibrosis marker are detected according to a non-invasive in vivo imaging technique (for example, modified luminescence detection, etc.). This detection method can be broadly utilized as a diagnostic marker for the treatment of fibrosis, without being limited to urologic diseases.

### (3-6) Diagnostic Marker for Hypoxia

Cells that express an HIF1α protein that is a marker that indicates the hypoxic conditions of living tissue are detected according to a non-invasive in vivo imaging technique (for example, modified luminescence detection, etc.). This detection method can be broadly utilized as a diagnostic marker that indicates the hypoxic conditions of tissues, without being limited to urologic diseases.

### (3-7) Improvement of Basal Pressure, Threshold Pressure, Micturition Pressure, Voiding Interval, Micturition Volume, or Residual Volume

When the cell structure of the present invention is transplanted to a neuropathic disease having such findings as the disappearance of peripheral nerves, a disease having such findings that acetylcholinesterase-positive cells are reduced or are not detected, or a disease having such findings that CGRP-positive afferent neurons are reduced or are not detected, the cells are engrafted and the neurons are infiltrated into the recipient tissues, so that the disease can be recovered. As a result, biochemical measurement values, namely, basal pressure, threshold pressure, micturition pressure, voiding interval, micturition volume, and residual volume are improved.

### EXAMPLES

Hereinafter, the present invention will be more specifically described in the following examples. However, these examples are not intended to limit the scope of the present invention.

### Example 1

### Animals

Twenty two female 10-week-old Sprague-Dawley (SD) rats (Japan SLC Inc., Shizuoka, Japan) were used as recipients. As donors for bone marrow cells, six 17-week-old Tg-SD rats (Japan SLC Inc.), which had been transfected with a green fluorescent protein (GFP), were used. All of the rats were bred with voluntarily feedable food and water under a 12-hour alternative light and dark cycle. After completion of each experiment, the rats were euthanized with an excessive amount of pentobarbital sodium solution (Kyoritsu Seiyaku Corporation, Tokyo, Japan). All of the animals were treated in accordance with the guidelines of National Institute of Animal Health and the guidelines approved by Animal Ethics Committee, School of Medicine, Shinshu University.

### Production of Radiation-Injured Bladder

The bladder was injured with radiation rays as follows.

The recipient SD rats were anesthetized with a pentobarbital sodium solution (Kyoritsu Seiyaku Corporation) in an amount of 40 mg/kg of body weight. Thereafter, the body of each rat was protected with a shield made of iron, except for a circle with a diameter of 1 cm, having the pubis as a boundary. Thereafter, the pubis region including the bladder (exposure region) was irradiated with radiation doses of 2 Gy, once a week, continuously for 5 weeks. After completion of the final radiation exposure, the rats were bred for 2 weeks. Three days before transplantation, the irradiated rats were subjected to an immunosuppressive treatment using cyclosporine (Novartis International AG, Basle, Switzerland) in an amount of 15 mg/kg of body weight, and 6α-methylprednisolone (Sigma-Aldrich, St. Louis, Mo.) in an amount of 2 mg/kg of body weight. Two weeks after the final radiation treatment, the treated rats were used as recipient animals.

### Production of Bone Marrow-Derived Cell Structure

Bone marrow-derived cells were prepared as follows.

Both femurs were collected from Tg-SD rats used as donors, into each of which a GFP gene had been introduced, and were then suspended in 10 ml of a medium, Dulbecco's Modified Eagle Medium (DMEM) High Glucose (Gibco, Thermo Fisher Scientific KK, Kanagawa, Japan), supplemented with 15% fetal bovine serum (BioWest, Nouaille, France) and 0.1% penicillin-streptomycin solution (Gibco). The cells were seeded on a 10-cm culture plate (ASAHI TECHNO GLASS, Shizuoka, Japan) coated with type I collagen and were then cultured for 7 days. When the cells became confluent, the adhering and proliferating bone marrow-derived cells were transferred into a 225-cm² culture flask (Asahi Techno Glass) coated with type I collagen, for subculture. In order to obtain a sufficient amount of cells, the subculture was carried out three times or four times.

After the bone marrow-derived cells had reached confluent as a result of the subculture performed three times or four times, the cells showed a relatively uniform spindle shape and were positive to STRO-1 as a mesenchymal cell marker. The bone marrow-derived cells were harvested, and were then suspended at a concentration of 4.0×10⁵ cells/ml in a spheroid-forming medium consisting of DMEM Low Glucose (Gibco) supplemented with 10% standard fetal bovine serum (BioWest) and 1.0% penicillin-streptomycin solution. In order to form spheroids, the cell suspension (4.0×10⁴ cells/0.1 ml) was seeded into each well of a 96-well U-shaped plate (Sumitomo Bakelite Co., Ltd., Tokyo, Japan), and were then cultured in a spheroid-forming medium at 37° C. in a 5% CO₂ atmosphere for 2 to 4 days. As a result, each of the 96 wells formed a single spheroid.

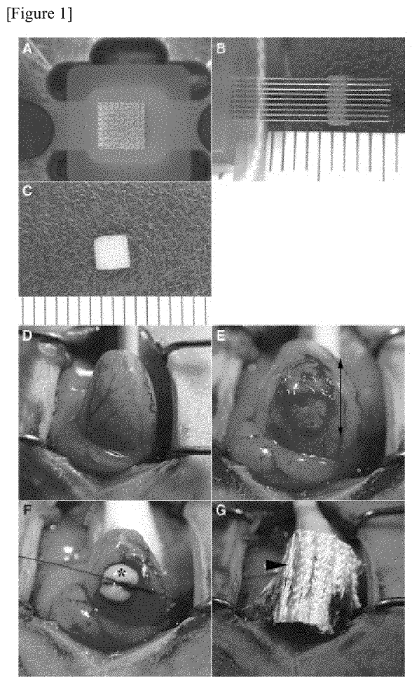

Subsequently, using a 3D bioprinting robot system, Regenova (Cyfuse Biomedical KK, Tokyo, Japan), bone marrow-derived cell spheroids were stacked to produce a three-dimensional structure. Regenova collected a spheroid from each of the 96 wells, and thereafter, the thus collected spheroids were inserted into a 9×9 microneedle array (approximately 5×5 mm, FIG. 1A). In the present example, three layers were formed on the microneedle array (height: 1 mm, FIG. 1B). In order to induce self-assembly, the formed spheroids, together with a spheroid-forming medium, were subjected to a perfusion culture at 37° C. in a 5% CO₂ atmosphere for 7 days. After completion of the perfusion culture, the microneedle array was removed from the self-assembled structure. The bone marrow-derived cell structure that was bio-processed from 243 spheroids had a 3 mm square and a height of 1 mm (FIG. 1C).

### Transplantation of Bio-Processed Bone Marrow-Derived Cell Structure

Two weeks after the final radiotherapy, the recipient rats were anesthetized by both a pentobarbital sodium solution and inhalation of 2% to 3% sevoflurane (Mylan Inc., Osaka, Japan). The irradiated bladder was exposed (FIG. 1D), and an incision with a size of approximately 5 mm was made on the anterior wall (FIG. 1E). The bio-processed structure was transplanted into the excised portion (rats, n=10), and then, was simply fixed with silk sutures 7-0 (FIG. 1F). The transplanted region was covered with an absorbable hemostatic agent (SURGICEL (registered trademark), Johnson and Johnson K.K., Tokyo, Japan) (FIG. 1G), so that the transplanted structure was prevented from transferring to other tissues around the bladder, such as adipose tissues. Finally, the bladder was returned to the pelvic cavity. Control rats were treated in the same manner as that for the above-described recipient rats, with the exception that the structure was not inserted into the 5-mm excised portion (false structure controls, n=12). Before the bladder was returned to the pelvic cavity, the excised portion was closed with sutures 7-0, and was covered with an absorbent hemostatic plug. All of the structure-transplanted rats and the false structure control rats were subjected to the immunosuppressive treatment (as described above) every week, and were further maintained for 2 weeks or for 4 weeks.

### Cell Measurement Test

Two and four weeks after the transplantation of the bio-processed structure (2 weeks, n=6; 4 weeks, n=4) or the sham surgery (n=6, in both periods), a bladder measurement test was carried out.

Two days before an intravesical pressure test, a polyethylene catheter was inserted into the bladder. The bladder measurement test was carried out for approximately 30 minutes on non-anesthetized and non-restrained rats that were each placed in a metabolic cage. A normal saline at room temperature was injected into the bladder at a rate of 10 ml/h through the catheter. The bladder contract and the micturition volume were simultaneously recorded on a pen-writing oscillograph. The following intravesical pressure parameters were measured: basal pressure, threshold pressure, micturition pressure (cmH₂O), voiding interval (minute), and micturition volume (ml). The residual volume (ml) was calculated by subtracting the micturition volume from the amount of the normal saline injected.

After completion of the bladder measurement test, the bladder was collected for histological and immunohistochemical examination (as described below). When significant adhesion of the adipose tissues was found, in order to avoid damage caused by an attempt to eliminate the adhering tissues, the bladder was collected together with the adipose tissues that adhered thereto.

### Histological and Immunohistochemical Investigation

The trimmed bladder was immobilized and was embedded in paraffin, and thereafter, the resultant was cut into continuous sections with a thickness of 5 μm. For the histological and immunohistochemical examination, the sections were stained with hematoxylin and eosin (HE), Masson's trichrome, enzyme-labeled acetylcholinesterase antibody (Medical & Biological Laboratories Co., Ltd., Nagoya, Japan), or Picrosirius Red.

For the immunohistochemical examination, the sections were stained with a GFP antibody (1:500, Mouse Monoclonal, Lifespan Biosciences, Inc., Seattle, Wash., USA). As a result, bone marrow-derived cells constituting a bio-processed structure were detected. The GFP antibody was detected by a secondary antibody consisting of Alexa fluor 488-bound donkey anti-mouse IgG (1:250, Molecular Probes, Eugene, Oreg., USA). Subsequently, the GFP antibody-stained sections were double-stained with an antibody against alpha-smooth muscle actin used as a smooth muscle cell marker (SMA, 1:100, Mouse Monoclonal, Progen Biotechnik GmbH, Heidelberg, Germany), or a calcitonin gene-related antibody. As a marker for afferent neurons, a CGRP peptide (1:500, Guinea Pig Monoclonal, Progen Biotechnik GmbH) was used.

These were detected by a secondary antibody consisting of donkey anti-mouse or anti-guinea pig IgG, conjugated with Alexa fluor 594 (1:250 in each case, Molecular Probes). Otherwise, other sections were stained with an SMA antibody, and was then detected by a secondary antibody consisting of Alexa fluor 594 (1:250, Molecular Probes)-conjugated donkey anti-mouse IgG.

The SMA antibody-stained section was double stained with collagen prolyl hydroxylase beta (P4HB, 1:50, Mouse Monoclonal, Novus Biological, Inc.) that was an enzyme essential for the synthesis of all collagens, and an antibody against hypoxia inducible factor 1α (HIF1α) that was a cell mediator in hypoxic response (1:50, Rabbit Polyclonal, Proteintech Group, Inc., Rosemont, Ill., USA).

The anti-P4HB antibody and the anti-HIF1α antibody were detected by a secondary antibody consisting of donkey anti-mouse or anti-rabbit IgG, conjugated with Alexa fluor 594 (1:250 in each case, Molecular Probes). The immunofluorescent section was stained, in comparison to nuclear staining with 4′,6-diamidino-2-phenylindole dihydrochloride (DAPI, 5 μg/ml, Molecular Probes).

### Statistical Analysis

The results were shown with a mean value±standard deviation. A statistical difference was determined using Excel (registered trademark) Statistical Program (ESUMI Co., Ltd., Tokyo, Japan). A comparison was made by non-repeated measure analysis of variance (ANOVA). A P value of less than 0.05 was determined to be statistically significant.

### <Results>

### Cell Structure of Bone Marrow-Derived Cells

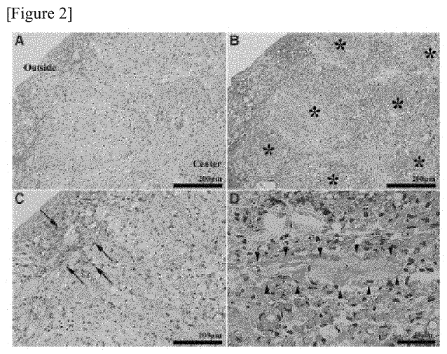

A cell structure that had not been used in transplantation was prepared, separately, and then, this cell structure was histologically examined. There were no disordered defects in the center of the structure (FIG. 2A). In the structure, the formed spheroids (FIG. 2B) were allowed to come into contact with cells proliferating in the spheroids (FIG. 2C) and/or in extracellular matrixes (FIG. 2D) secreted from the cells, so that the spheroids were self-assembled. Before transplantation, the cells in the structure were positive to the GFP antibody, but were negative to the SMA antibody or the CFRP antibody.

Survival of Cell Structure Transplanted into Irradiated Bladder

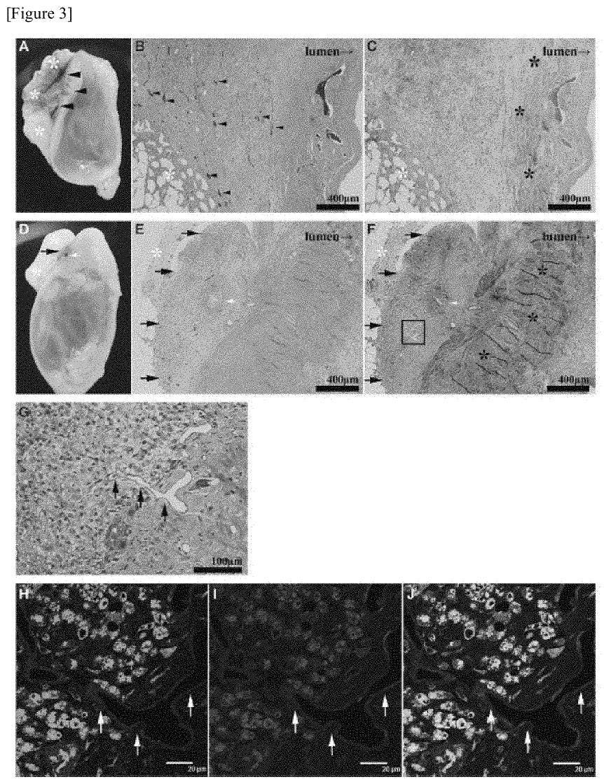

Two weeks after the transplantation of a bio-processed structure and the control surgery of a false structure, the control bladder did not show at all the recovery of the incision made on the anterior wall of the urinary bladder (FIG. 3A). Moreover, the injured region had a large nucleus, as with microphages and/or eosinophilic leukocytes, and also had a large number of inflammatory cells whose cytoplasm was stained by eosin staining (FIG. 3B). Furthermore, the smooth muscle layer was thin, and was sparsely distributed (FIG. 3C). In contrast, the transplanted bio-processed structure was easily recognized in the region of the anterior wall of the urinary bladder (FIG. 3D). The transplanted structure survived in the recipient tissues, and almost no inflammatory cells were found in the bladder tissues around the transplanted region (FIG. 3E).

A different smooth muscle layer was present in the bladder tissues near the transplanted structure (FIG. 3F). Moreover, blood vessels were found in the boundary between the transplanted cell structure and the recipient tissues (FIG. 3G). The smooth muscle cells in the vascular wall were negative to the GFP antibody (FIG. 3H). Thus, the blood vessels were derived from the recipient tissues, and were extended into the transplanted cell structure. The bone marrow-derived cells constituting the cell structure that were simultaneously positive to both the GFP (FIG. 3H) and SMA (FIG. 3I) antibodies were differentiated into smooth muscle cells, such that the cells surrounded the enlarged blood vessels (FIG. 3J). The double-positive differentiated smooth muscle cells were broadly distributed, but those cells did not form a cell cluster (FIG. 3J).

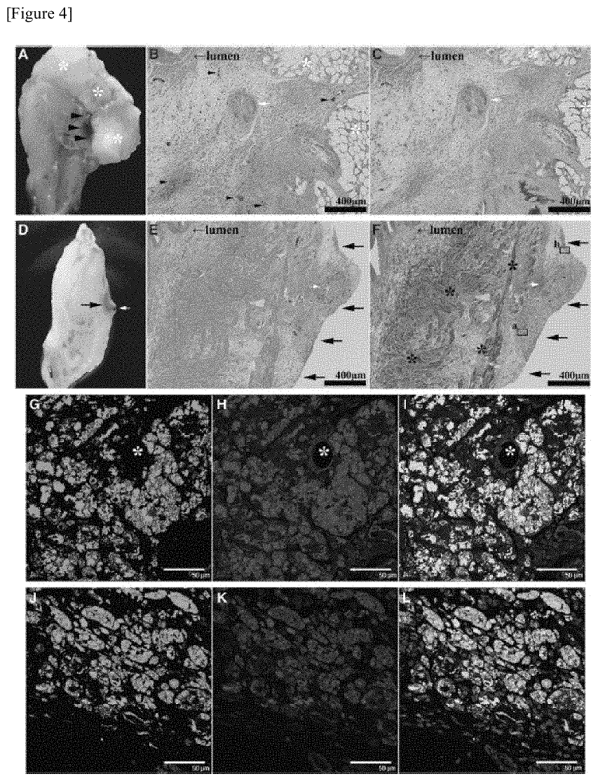

Four weeks after the sham surgery, the surgical site was excised and confirmed. As a result, the surgical site was hardly recovered, compared with the surgical site 2 weeks after the surgery (FIG. 4A). The control surgical region had a large number of inflammatory cells (FIG. 4B), and the smooth muscle layer was dissociated (FIG. 4C). In contrast, the transplanted cell structure was easily identified (FIG. 4D), and was well integrated into the recipient bladder wall (FIG. 4E). The recipient bladder wall around the transplanted region had a clear smooth muscle layer (FIG. 4F), which was not observed in the region after completion of the sham surgery (FIG. 4C). Furthermore, the reconstructed smooth muscle layer was almost the same as the non-irradiated normal bladder. In the transplanted cell structure, differentiated smooth muscle cells, which surrounded blood vessels derived from the recipient tissues, formed a cluster structure (FIGS. 4G to I). In addition, even outside of the transplanted structure, there was a relatively small cluster structure consisting of the differentiated smooth muscle cells (FIGS. 4J to L).

### Histological Change in Recipient Bladder Tissues

The present inventor has conducted immunohistochemical examination and histological examination, and has observed bladder tissues around the boundary between the recipient tissues and the transplanted cell structure. In addition, the present inventor has also observed bladder tissues around the excised region of the bladder subjected to a sham surgery.

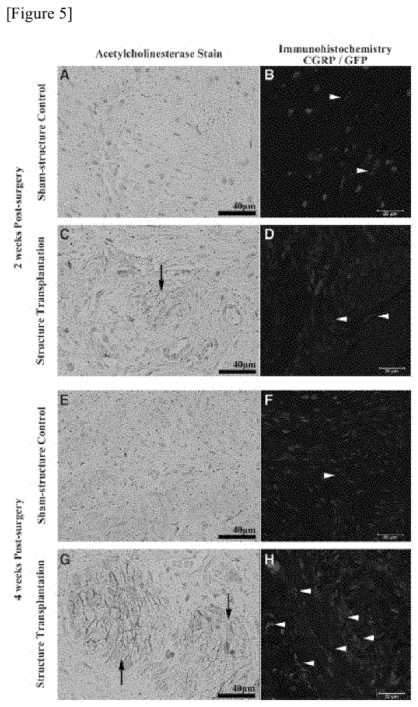

Two weeks after the control surgery, acetylcholinesterase-positive cells (FIG. 5A) and CGRP-positive afferent neurons (FIG. 5B) were hardly present. On the other hand, in a cell structure-transplanted group, several acetylcholinesterase-positive cells were present two weeks after the transplantation (FIG. 5C). In contrast to the number of the acetylcholinesterase-positive cells, the number and distribution of the CGRP-positive afferent neurons were the same as those for the bladder tissues in the same period as the transplantation (FIG. 5D).

In the sham surgery group, even 4 weeks after the surgery, acetylcholinesterase-positive cells were not clearly shown (FIG. 5E). In addition, in comparison to the control bladder two weeks after the sham surgery (FIG. 5B), only a small number of the CGRP-positive afferent neurons were found (FIG. 5F). However, 4 weeks after the transplantation of the cell structure, the recipient bladder tissues had a large number of the acetylcholinesterase-positive cells (FIG. 5G) and the CGRP-positive cells (FIG. 5H), and the number of these cells was significantly higher than the number of the cells clearly observed in the transplanted recipient bladder 2 weeks after the surgery.

Besides, both 2 weeks and 4 weeks after the surgery, differentiation of the bone marrow-derived cells constituting the structure into CGRP-positive cells (afferent neurons) was not found in either the transplanted structure or the recipient bladder tissues. It was unknown whether the neurons were induced from the periphery as a result of the transplantation of the structure, or the cells that barely survived after irradiation were recovered and proliferated. Anyway, it was confirmed that the neurons of the recipient were regenerated (recovered) as a result of the transplantation of the structure, and thus that the bladder functions were improved.

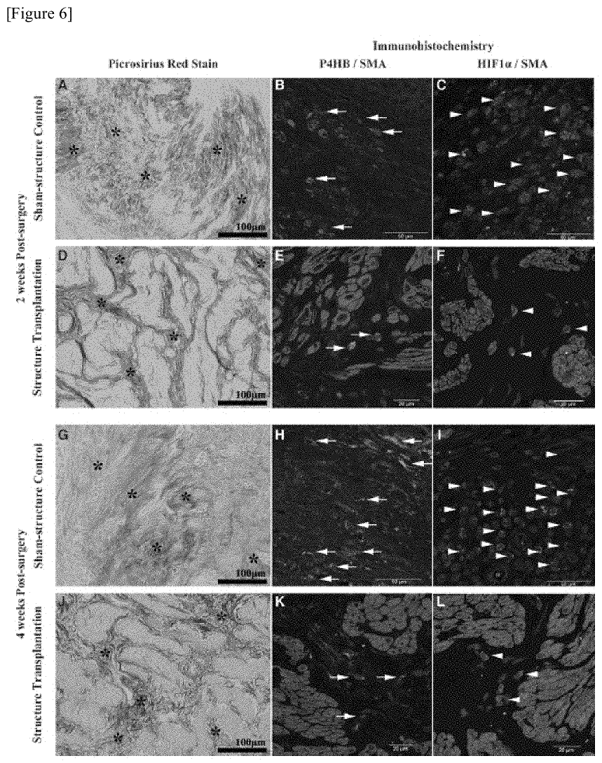

Two weeks after the sham surgery, collagen fibers were infiltrated into the spaces of extracellular matrixes comprising injured smooth muscle layers (FIG. 6A). The cells in the collagen fiber region expressed P4HB serving as a fibrosis marker (FIG. 6B). Moreover, the control bladder tissues had a large number of HIF1α-positive cells serving as a marker for hypoxia (FIG. 6C). In contrast, two weeks after the transplantation of the cell structure, collagen fibers in the recipient bladder tissues were distributed among clusters of the reconstructed smooth muscle cells (FIG. 6D). Among the clusters of the smooth muscle cells, a small number of P4HB-positive cells (FIG. 6E) and a small number of HIF1α-positive cells (FIG. 6F) were present. However, the numbers of these cells were smaller than those of the cells in the control bladder tissues (FIG. 6B and FIG. 6C).

Four weeks after the sham surgery, collagen fibers are significantly enlarged, and occupied the extracellular space in the absence of a smooth muscle layer (FIG. 6G). Several regions having a smooth muscle layer had a large number of P4HB-positive cells (FIG. 6H) and a large number of HIF1α-positive cells (FIG. 6I).

In contrast, collagen fibers in the recipient bladder tissues 4 weeks after the transplantation of the cell structure were integrated among the clusters of smooth muscle cells (FIG. 6J). Distribution of these collagen fibers was almost the same as that in the recipient bladder tissues 2 weeks after the transplantation (FIG. 6D). Four weeks after then transplantation of the cell structure, the recipient bladder tissues had a small number of P4HB-positive cells (FIG. 6K) and a small number of HIF1α-positive cells (FIG. 6L) in the collagen fibers. The numbers of these cells were smaller than those of the cells in the control bladder tissues in the same period after the surgery (FIG. 6H and FIG. 6I).

### Recovery of Bladder Functions

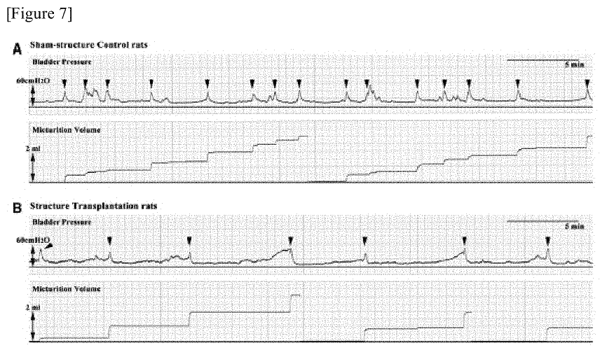

In the intravesical pressure test conducted 2 weeks after the sham surgery or the transplantation of the cell structure, the micturition patterns were similar to each other. Four weeks after the sham surgery, the voiding interval of the control rats was less than 5 minutes (irregular voiding interval), and the micturition volume thereof was less than 1 ml, which showed significant symptoms of frequent micturition (FIG. 7A). However, in the rats 4 weeks after the transplantation of the cell structure, the voiding interval was approximately 5 minutes, and the micturition volume was approximately 1 ml, which showed regular micturition (FIG. 7B). Accordingly, in the rats having a radiation-injured bladder, into which the cell structure had been transplanted, the frequent micturition symptoms 4 weeks after the surgery were improved in comparison to the control rats subjected to the sham surgery, and the number of micturitions was also reduced.

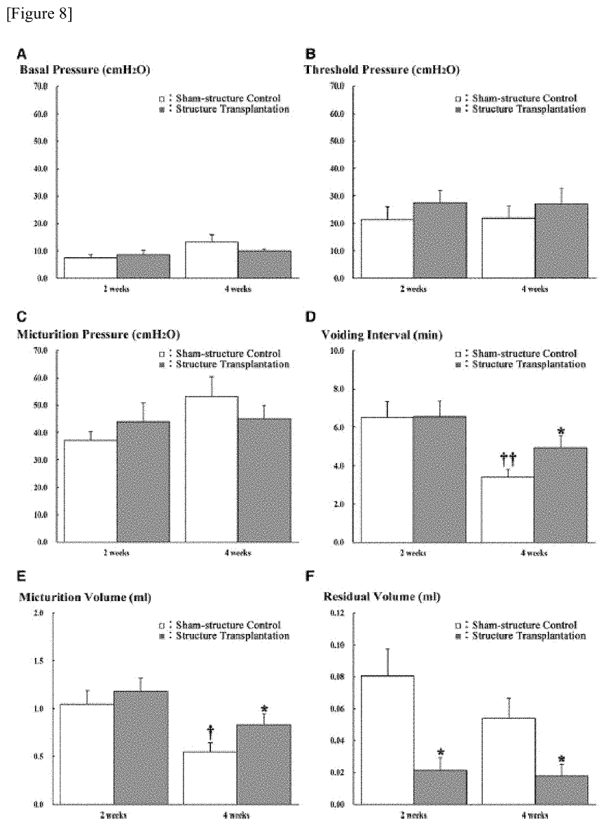

Individual micturition parameters were assumed from the intravesical pressure chart. Two weeks after the sham surgery or the transplantation surgery of the cell structure, basal pressure, threshold pressure, micturition pressure, voiding interval, and micturition volume were measured. Between the sham surgery control group and the cell structure transplantation group, there was no significant difference (FIGS. 8A to E). However, the residual volume in the cell structure-transplanted rats (0.02±0.01 ml) was significantly lower than that in the control group (0.08±0.02 ml, P<0.05, FIG. 8F). Thus, the micturition efficiency of the cell structure-transplanted rats was improved compared with the rats in the control group.

Four weeks after the sham surgery, the basal pressure (FIG. 8A), the threshold pressure (FIG. 8B), the micturition pressure (FIG. 8C), and the residual volume (FIG. 8F) were not changed from the values in the sham surgery group 2 weeks after the sham surgery.

However, regarding the voiding interval (FIG. 8D) and the micturition volume (FIG. 8E), the voiding interval in the control rats 4 weeks after the sham surgery was 3.40±0.43 minutes (FIG. 8D), and the micturition volume was 0.55±0.09 ml (FIG. 8E). On the other hand, the voiding interval in the control rats 2 weeks after the sham surgery was 6.52±0.81 minutes (P<0.01, FIG. 8D) and the micturition volume was 1.04±0.14 ml (P<0.05, FIG. 8E). Accordingly, these values 4 weeks after the sham surgery were significantly lower than the values 2 weeks after the sham surgery. Regarding the voiding interval (FIG. 8D) and the micturition volume (FIG. 8E), the values 4 weeks after the transplantation of the cell structure were not significantly deteriorated from the values 2 weeks after the transplantation of the cell structure (FIG. 8D and FIG. 8E).

In terms of the basal pressure, the threshold pressure, and the micturition pressure 4 weeks after the sham surgery group or the transplantation of the cell structure, there was no significant difference between the sham surgery group and the cell structure transplantation group (FIGS. 8A to C). However, in terms of the voiding interval (4.93±0.63 minutes, FIG. 8D) and the micturition volume (0.83±0.12 ml, FIG. 8E) in the cell structure-transplanted rats 4 weeks after the surgery, the values in the cell structure-transplanted rats were significantly higher than the values in the control rats in the same period after the surgery (P<0.01 and P<0.05, respectively). In addition, the residual volume in the cell structure-transplanted rats 4 weeks after the surgery (0.02±0.01 ml) was significantly smaller than the residual volume in the false structure control rats in the same period after the surgery (0.05±0.01 ml) (P<0.05, FIG. 8F).

These micturition parameters in the cell structure-transplanted bladder were almost the same as those in the normal bladder that had not been irradiated with radioactive rays. These results demonstrated that the cell structure-transplanted rats are partially prevented from reductions in both urine collection function and micturition efficiency that occur in radiation-injured bladders.

### <Consideration>

The present inventor has studied a method of assisting the structural and functional recovery of the lower urinary tract that is mainly composed of the bladder and the urethra. Then, the present inventor has applied a tissue engineering methodology of utilizing a combination of biochemical factors based on cells, biological materials, and microenvironments in the target tissues and organs of a recipient. At current, biofabrication realized by a 3D bioprinter has been reported as a new biotechnology. Accordingly, we have incorporated this new biotechnology into our tissue engineering methodology. This methodology is referred to as “next-generation tissue engineering.”

By using the 3D bioprinting robot system, Regenova, a self-assembled tissue-like structure consisting of bone marrow-derived cells was biofabricated. The cell structure of the present invention has several advantages, which are not found in either a cell injection method or a cell sheet method. Firstly, the present cell structure has a thickness and a strength that are sufficient for facilitating the handling for transplantation. Secondly, the present cell structure can be directly transplanted into recipient tissues. Thirdly, the 3D conformation imitates naturally occurring tissues, and provides an intracellular contract for promoting self-assembly. Finally, the present cell structure has biocompatibility that is higher than that of an artificial material.

Regarding the effect of the cell structure on recipient tissues, a paracrine effect on the microenvironment of the recipient tissues adjacent to the cell structure is expected. Accordingly, a histological change at the boundary surface between the structure to be transplanted and the recipient tissues is important. The most important result is that the transplanted structure survived in the recipient tissues, and that blood vessels grew and extended from the adjacent recipient tissues into the cell structure.

While surrounding the extended blood vessels, the bone marrow-derived cells in the cell structure were differentiated into smooth muscle cells. Four weeks after the transplantation, those smooth muscle cells form a cluster thereof at the outer edge of the extended blood vessels surrounding them and the transplanted structure. In the transplanted cell structure, HIF1α-positive cells exhibiting hypoxia were sparsely distributed. Hence, the microenvironment surrounding the blood vessels extended from the recipient tissues supports differentiation of smooth muscle cells from the bone marrow-derived cells and cluster formation of the smooth muscle cells, and it shows that the smooth muscle cells have been differentiated from the bone marrow-derived cells.

It has been reported that a signal pathway including the expression of hypoxia-dependent HIF1α causes fibrosis in an injured bladder (Ekman, M. et al., Lab Invest 94, 557, 2014, Iguchi, N. et al., Am J Physiol Renal Physiol, 313, F1149, 2017, Wiafe, B. et al., In Vitro Cell Dev Biol Anim 53, 58, 2017.). However, at the time points that were 2 weeks and 4 weeks after the surgery, the number of HIF1α-positive cells as hypoxia markers was small in the recipient tissues of a cell structure-transplanted bladder, in comparison to that in a false structure control. In addition, significant fibrosis was not developed in the structure-transplanted bladder, or the structure-transplanted bladder comprised a large number of P4HB-positive cells.

The presence of the cell structure and the relevant growth of blood vessels from the surrounding tissues into the cell structure create an optimal microenvironment, and reduce or eliminate hypoxia that relates to wound. Consequently, the HIF1α pathway is activated only transiently and/or to the minimum. Accordingly, it is considered that decomposition of extracellular matrix collagen mediated by the P4HB pathway is limited. This explains that fibrosis is significantly reduced (improved) in a bladder into which the cell structure has been transplanted. Moreover, the histological findings of such a cell structure-transplanted bladder were similar to the histological findings of a normal bladder that has not been irradiated with radioactive rays.

Furthermore, as shown in the previous studies, it was shown that the transplantation of the cell structure into the irradiated bladder induces the recovery of the bladder functions (Imamura, T. et al., Tissue Eng Part A 18, 1698, 2012, Imamura, T. et al., Tissue Eng Part A 21, 1600, 2015.). Four weeks after the transplantation, the cell structure-transplanted rats did not exhibit significant frequent micturition symptoms. The voiding interval and the micturition volume were higher in the cell structure-transplanted rats than those in control rats. Moreover, both 2 weeks and 4 weeks after the transplantation, the residual volume in the cell structure-transplanted rats was smaller than the residual volume in the control rats. The transplantation of the cell structure improved both urine collection function and micturition efficiency, and recovered them to the levels of a normal bladder. Furthermore, these results suggest that the transplanted structure may suppress progressive irradiation damage. It is considered that the improvement or suppression of progressive irradiation damage is associated with alleviation of radiation-induced frequent micturition symptoms.

Differing from the advantages of the present cell structure that exceeds a single-type cell direct injection method and a cell sheet patch transplantation method, the cell structure enables the delivery of a larger number of cells to the target site.

With regard to the construction of the cell structure, 243 spheroids were inserted into an area from the middle part to the upper part of a 9×9 microneedle array. Each spheroid was formed with 4×10⁴ cells. As a result, the number of cells in each cell structure was approximately 1×10⁷ cells (243 spheroids×4×10⁴ cells/spheroid=approximately 1×10⁷ cells). This number of cells is approximately 100 times greater than the number of cells used in either the cell injection method or the cell sheet method.

Even two weeks after the transplantation, which was a half of the period in the previous studies conducted by the present inventor, the bladder functions of the cell structure-transplanted rats, except for the residual volume, were not changed from those of the sham surgery control rats. Accordingly, strictly speaking, the recovery 2 weeks after the transplantation does not seem to be associated with the number of the transplanted bone marrow-derived cells. The data provided by the present inventors suggest that, regarding reconstruction of functional tissues, a certain recovery period, namely, at least 4 weeks, should be required for cell replication, differentiation, and tissue construction, in the case of the experimental models used herein. During such a recovery period, the transplanted cell structure is able to replace and substitute for damaged tissues, so that the present cell structure could provide regeneration effects, which had not been elucidated by the direct injection method and the cell sheet method.

In conclusion, for the biofabrication of a cell structure consisting of bone marrow-derived cells, the present inventor has used a 3D bioprinting robot system. The cell structure survived after it had been transplanted into the irradiated rat bladder, and blood vessels were infiltrated from the recipient tissues adjacent to the cell structure into the cell structure. The bone marrow-derived cells constituting the cell structure were differentiated into smooth muscle cells, which then formed a cluster thereof. Although the transplanted cells were not differentiated into neurons, the regenerated neurons were present in the recipient bladder tissues. In the bladder tissues into which the cell structure had been transplanted, significant fibrosis associated with HIF1α-positive cells and P4HB-positive cells was not developed. Four weeks after the transplantation of the cell structure, the frequent micturition symptoms of the rats were improved, and the residual volume was reduced. Therefore, the present cell structure will become a great tool for treating patients having severe lower urinary tract symptoms that are caused by the damage of the bladder.

1. A material for inducing nerve regeneration in a transplantation site, wherein the material comprises a cell structure having a thickness of at least 300 μm that is constructed by stacking the spheroids of bone marrow-derived cells, adipose tissue-derived cells, dental pulp-derived cells, amnion-derived cells, placenta-derived cells, umbilical cord-derived cells, or umbilical cord blood-derived cells, on a needle-shaped body arranged on a substrate.

2. The nerve regeneration-inducing material according to claim 1, wherein the transplantation site is a tissue or an organ comprising a smooth muscle layer.

3. The nerve regeneration-inducing material according to claim 2, wherein the tissue or organ comprising a smooth muscle layer is at least one selected from the group consisting of bladder, ureter, urethra, penis, uterus, vagina, spermatic duct, and fallopian tube.

4. The nerve regeneration-inducing material according to claim 1, which further provides at least one selected from the group consisting of regeneration of microvessels, normalization of collagen fibers, the improvement of hypoxia, and the improvement of basal pressure, threshold pressure, micturition pressure, voiding interval, micturition volume, and residual volume in the bladder.

5. A material for recovering nerve function in a transplantation site, wherein the material comprises a cell structure having a thickness of at least 300 μm that is constructed by stacking the spheroids of bone marrow-derived cells, adipose tissue-derived cells, dental pulp-derived cells, amnion-derived cells, placenta-derived cells, umbilical cord-derived cells, or umbilical cord blood-derived cells, on a needle-shaped body arranged on a substrate.

6. The function-recovering material according to claim 5, wherein the transplantation site is a tissue or an organ comprising a smooth muscle layer.

7. The function-recovering material according to claim 6, wherein the tissue or organ comprising a smooth muscle layer is at least one selected from the group consisting of bladder, ureter, urethra, penis, uterus, vagina, spermatic duct, and fallopian tube.

8. The function-recovering material according to claim 6, which further provides at least one selected from the group consisting of regeneration of microvessels, normalization of collagen fibers, the improvement of hypoxia, and the improvement of basal pressure, threshold pressure, micturition pressure, voiding interval, micturition volume, and residual volume in the bladder.
